(19) United States
(12) Patent Application Publication
(10) Pub. No.: US 2018/0303174 A1
(43) Pub. Date: Oct. 25, 2018

(54) Three Fingered Gaming Helmet

(71) Applicant: Antonio Celester, Framingham, MA (US)

(72) Inventor: Antonio Celester, Framingham, MA (US)

(21) Appl. No.: 15/932,721

(22) Filed: Apr. 13, 2018

Related U.S. Application Data
(60) Provisional application No. 62/489,625, filed on Apr. 25, 2017.

(51) Int. Cl. A41D 19/015; A63F 13/98; A61F 5/01; A61F 13/10

(52) U.S. Cl. CPC A41D 19/015; A61F 13/104; A61F 5/0118; A63F 13/98

(57) ABSTRACT

The three-fingered gaming helmet, is the new level of slip on protective safety for gamers, who use a multifunction hand-held controller, with a desire to have longer playing sessions, without injuries.

### BACKGROUND OF THE INVENTION

Video gaming has evolved from a simple game played at home, using a hand held gaming controller, that interacts to generate a visual feedback on a video device, such as a TV monitor, to a more advanced system, that requires an advanced hand held controller and eye coordination, in order to master certain technics, by fingered movements with a hand held multifunction controller.

There are four basic areas, while using the gaming controller during play, which comes in direct contact with the user:

a. The palm of the hands,

b. The thumbs,

c. The forefingers, and

d. The middle fingers.

Gaming often require that the person using a hand-held controller, to act with quickness, using the multifunction's inputs, movement commands, in a way and manner that brings the most success to the user. Each game played may need a different motion or movement, creating direct user contact, in the four basic areas of the players hand and finger areas as mentioned.

These direct contacts through using the hand-held controller, while playing, creates friction, through continued use and multiple movements, in the areas of the user's palms and three finger areas. Injuries start to develop, due in part to the amount of action movements needed in each game played and due in part to excessive use and direct contact with a hand-held gaming controller.

These injuries include, but not limited to blistering, Calluses, the Harding of the affected skin areas, fingers, and hand, which in some cases deformities of the hand and fingers of hand-held controller are reported.

Users would continue to play, despite injuries, develop a more painful condition and further the injuries by playing through the pain and compounding the damage to their finger and thumbs areas.

The need for a protective barrier, between the hand-held controller and the user, is the birth of the three-fingered gaming helmet.

This invention named a “helmet” to denounce its need for safe play. It is a safety device and protection device, used while playing and using a hand-held gaming controller.

The three-fingered gaming helmet gives protection from calluses, blistering and other injuries, while also giving worry free extended playing time. The most protection is giving in the areas that are most likely to develop injuries by user play. The three-fingered gaming helmet features internal reinforced barrier material in the areas of the thumb, forefinger, and middle fingers, to protect these shared areas of injuries, hence the name “helmet”.

The present invention uses thin materials to insure the greatest comfort, while creating the necessary barrier, between the user and the gaming controller, without taking away the user feel or ability to execute quick and exact movements, during game play.

An added benefit to the three-fingered gaming helmet, the materials can be interchangeable for other gaming therapeutic purposes, used during game play for certain users.

For example, compressed materials can offer benefits to users with arthritis or carpel tunnel conditions. The three-fingered gaming helmet comes in assorted sizes to accommodate and protect the many users of hand-held gaming controllers.

### SUMMARY OF THE INVENTION

The first purpose of the invention is to “protect” hand-held controller gaming users from injuries.

The second purpose is to allow “extended playing time” of gaming users, who game with hand-held controllers, without the worry of injuries.

Until now there was no such protection from game playing injuries, due to the use of hand-held multifunction gaming controllers. There was no protection that allowed extended playing time, without the worry of injuries, with garners playing through pain, causing further damage.

This invention protects all hand-held controller gaming users, regardless of age, gender, or experience in gaming. A user can simply slip on this protection before use and have endless gaming sessions, from amateur levels to professional competitions.

### DRAWINGS

### A Brief Description of the Photos

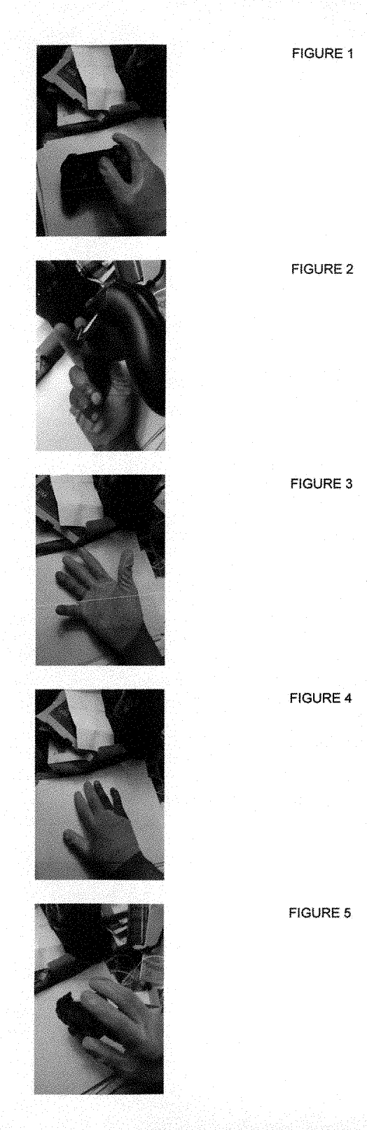

FIG. 1. Shows the helmet being worn and shows the right hand cut out, top view, with a sample type material, irrespective to the invention, demonstrating how a user would wear the right hand helmet, the dark orange demonstrate the areas where the inner barrier, the helmet, would be formed, with reinforced material, in order to give the greatest protection to the user.

FIG. 2. Shows the helmet worn and shows the right hand cut out, bottom side view, looking from left to right of the controller. The dark orange depicts the reinforced protected area in the right-hand palm and shows the two free fingers, that do not come into play, as well as the reinforced portions on the remaining three fingers.

FIG. 3. Shows the helmet worn and shows the opened right hand cut out, faced palm up, showing the complete reinforced area, the helmet, used in this invention and lined inside the material and used as the barrier to protect the user from injuries.

FIG. 4. Shows the helmet worn and shows the right hand cut out, faced down, back side, showing the areas not offering protection and therefore excluded of reinforced materials. Material used on the back side would allow hand ventilation and comfort for the user.

FIG. 5. Shows the helmet worn and shows the right hand, looking from the right side of the controller to the left, arming the three areas of the controller right side functions, where the user comes in protected contact with the three areas of the device, while wearing the three-fingered helmet.

The foregoing description of specific embodiments of the present invention have been presented for purposes of illustration and description. They are not intended to be exhaustive or limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I. The three-fingered gaming helmet, comprising of three fingered covering, of the thumb, the forefinger and the middle finger, joined together with a covering of the palm, lined with an internal protective reinforced barrier material, on the full face up, palms and the three fingers called, the “Helmet” and continued internal protective reinforced barrier material, lined around the fingered areas.

II. The three-fingered gaming helmet of claim I further comprising: Therapeutic materials, compressed or not, with or without risk supports, wherein these components are a variation of design, in order to aid users with disabilities or debilitating medical conditions and who use multifunction hand held controllers for gaming.
